(19) United States
(12) Patent Application Publication
(10) Pub. No.: US 2022/0008344 A1
(43) Pub. Date: Jan. 13, 2022

(54) SUGAR-COATED SOLID FORMS HAVING IMPROVED STABILITY

(71) Applicant: ROQUETTE FRERES, Lestrem (FR)

(72) Inventors: Philippe LEFEVRE, Haverskerque (FR); Grégory LE BIHAN, Cambrin (FR); Sébastien CROQUET, Merville (FR)

(21) Appl. No.: 17/309,670

(22) PCT Filed: Dec. 17, 2019
(86) PCT No.: PCT/FR2019/053106

§ 371 Date: Jun. 14, 2021

(30) Foreign Application Priority Data
Dec. 17, 2018 (FR) 18 73051

(51) Int. Cl. A61K 9/28

(52) U.S. Cl. CPC A61K 9/2826; A61K 9/2893

(57) ABSTRACT

The present invention relates to novel sugar-coated solid forms having improved stability, in particular a superior humidity resistance, and to a sugar-coating method which is particularly useful for the preparation of same.

The object of the present invention is novel sugar-coated solid forms having improved stability, in particular greater humidity resistance, as well as a sugar-coating method particularly useful for the preparation of same.

### FIELD OF THE INVENTION

Solid forms such as powders and tablets are called hygroscopic when they are composed of substances having a tendency to absorb humidity. This humidity affects the weight of the products, their solidity and their quality. By protecting these humidity-sensitive ingredients, unnecessary losses caused by a too high relative humidity level in their environment are avoided.

To this end, it is possible to act on several levels: humidity control in the production, transport and storage areas, specific packaging, modification of the pharmaceutical form.

As regards the pharmaceutical form, conventional protection techniques consist in coating or including these solid cores made up of hygroscopic substances within a protective envelope, for example by means of encapsulation techniques (in hard capsules or soft capsules), film-coating techniques or sugar-coating techniques.

Sugar-coating is an operation used especially in confectionery or pharmaceutics, which consists in creating a more or less hard or soft crystallized coating on the surface of solid or powdery products. The coating of the solid form (core) is carried out in a tank rotating about its axis and called a coater, inside which there is a plurality of cores forming a moving mass, on the surface of which the constituent materials of the future envelope (“sugar-coating liquid or syrup”) is distributed in the liquid state. The hard and crystalline coating is obtained by applying this liquid and evaporating the water provided by it.

In film-coating, a composition based on polymers having film-forming properties is applied instead of a composition of crystallizable materials. This composition forms a thin film that can act as a humidity barrier. The polymers selected for this purpose are synthetic polymers such as polyvinyl alcohol (PVA) and derivatives thereof. This method is faster than sugar-coating, but does not provide the characteristic appearance and texture of sugar-coated tablets.

The sugar-coating syrup is mainly made up of one or even several crystallizable materials, and also conventionally contains binding agents such as gum arabic or gelatin, colorants, opacifiers such as TiO₂, mineral fillers such as talc, silica, calcium carbonate, intense sweeteners, flavorings, vitamins and active ingredients.

As a crystallizable materials, mannitol could be an ideal candidate for creating a humidity barrier due to its low hygroscopicity. In addition, mannitol has a low reactivity with respect to active ingredients, which makes it an excipient of choice in pharmaceutics.

However, mannitol coating faces technical difficulties which cause this substance to not be currently used in sugar-coating. Although crystallizable, mannitol has low solubility in water. However, in the sugar-coating methods, the sprayed syrup must have a high content of crystallizable materials in order to be able to form a sugar-coating layer. For conventional maltitol or sucrose coating, at least 70% dry material is required. Such dry material cannot be obtained with mannitol, even at high temperature.

Interestingly, mannitol coating compositions and film-forming polymers of synthetic origin (polyvinyl alcohol (PVA) and derivatives thereof), exhibiting a certain stability, have also been disclosed (U.S. Pat. No. 8,846,089 (WAKUNAGA PHARMACEUTICALS). However, the stability of the solid forms thus coated still needed to be improved.

There was therefore an unsatisfied need to provide means for coating solid forms making it possible to obtain improved stability.

### SUBJECT MATTER OF THE INVENTION

The object of the present invention is therefore to provide coated solid forms having good stability, in particular good humidity resistance, and which are as effective, or even better than those from the prior art.

Another preferred object of the present invention is to provide solid forms whose coating does not require the use of materials of synthetic origin such as PVA.

### DESCRIPTION OF THE INVENTION

The Applicant has succeeded in producing solid forms coated with a mannitol-based composition, which has a very good humidity resistance. These coating compositions do not require the use of film-forming polymers of synthetic origin such as PVA, and furthermore exhibit improved stability.

This coating is characterized in that it uses a combination of mannitol and fatty acids, in particular stearic acid.

The Applicant has achieved this by developing a method for sugar-coating solid forms, characterized in that it comprises:
a step (a) of spraying a sugar-coating liquid onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid;
a step (c) of collecting the resulting sugar-coated solid forms; said sugar-coating liquid comprising mannitol and fatty acids.

The resulting coating provides an excellent humidity barrier. As demonstrated in the examples below, this barrier effect is much better than that obtained with a coating composition using mannitol and PVA as used in the aforementioned U.S. Pat. No. 8,846,089 (WAKUNAGA PHARMACEUTICALS).

As can be seen from the Examples below and in particular from photographs taken on cross sections of coated tablets, it seems that the structure of the coating layer obtained in accordance with the aforementioned U.S. Pat. No. 8,846,089 is different from that obtained with the technology of the invention. Without wishing to be bound by any theory, the Applicant is of the opinion that this structural difference could explain this difference in performance.

Advantageously, the coating composition of the invention does not necessarily require the use of titanium dioxide. Indeed, the solid forms thus coated, in particular those of white color, do not yellow over time.

The method of the invention applies to various solid forms (tablets, chewing gums, etc.) and offers multiple possibilities for manufacturers, and in particular that of being able to produce sugar-coated solid forms comprising humidity-sensitive substances.

In the method of the invention, drying and spraying are performed concomitantly. Surprisingly, despite the continuous humidification of the cores, these do not erode.

This is particularly surprising since it has hitherto been accepted in sugar- coating that spraying must necessarily be stopped for a not-insignificant time during the method. Otherwise, the cores are continuously humidified, so that the substance used for the coating cannot crystallize, the solid form erodes and degrades, and the cores adhere to each other (sticking phenomenon). However, this phenomenon is not observed with the method of the invention, even on very hygroscopic cores, that is to say attracting and holding large water amounts.

Thus, and unlike conventional sugar-coating, the method of the invention does not require the sugar-coating liquid to have a high dry material content (of at least 70% for conventional maltitol coating). This also goes against general knowledge of the art for the same reasons as those mentioned in the preceding paragraph (significant humidifying of the cores).

The fact of being able to use low dry material has a certain number of advantages. Firstly, it gives the possibility of using coating liquids at low temperatures, and therefore of introducing thermolabile ingredients into these liquids or into the core compositions to be sugar-coated. Indeed, if high temperatures are conventionally used in sugar coating, it is to allow the sugar-coating solution to have a high content of crystallizable materials in solution. Since this high dry material is not necessary in the method of the invention, the temperature of the sugar-coating liquid can be lowered.

Secondly, this also makes it possible to carry out sugar-coating operations with crystallizable materials having a low solubility in water, which is precisely the case with mannitol.

The method developed by the Applicant is moreover much more flexible than conventional sugar-coating methods, and may be particularly simple in its implementation.

Finally, the method of the invention does not necessarily require large modifications of its parameters during its execution (drying temperature, formulation of the sugar-coating liquid, etc.), and can be implemented in relatively simple and space-saving equipment.

Finally, with regard to the method of the invention, mention may be made of French patent application No. 1755557 in the name of the Applicant. This patent application, not yet published, discloses a method close to that of the present invention, and advantageously makes it possible to sugar coat solid forms with mannitol. However, that patent application does not mention the use of fatty acids.

### SUMMARY OF THE INVENTION

The first object of the present invention is a solid sugar-coated form, characterized in that it comprises at least one sugar-coating layer comprising mannitol and fatty acids.

An object of the present invention is also a solid form sugar-coating method, particularly useful for the preparation of sugar-coated solid forms of the invention, characterized in that it comprises:

a step (a) of spraying a sugar-coating liquid onto a bed of moving cores, said cores being placed in a chamber equipped with a perforated rotary drum, and said spraying being ensured by means of at least one compressed air nozzle;
at least one step (b) concomitant with step (a) of drying said sprayed sugar coating liquid;
a step (c) of collecting the sugar coated solid forms thus obtained;
said sugar coating liquid comprising mannitol and fatty acids.

An object of the present invention is also the use of a combination of mannitol and fatty acids for coating solid forms, in particular in a sugar-coating method in accordance with the invention, in order to improve the stability of said solid forms.

### FIGURES

FIG. 1

[FIG. 1]: Fracture of the sugar-coated layer—Scanning Electron Microscopy x135, Sugar-coating liquid Man_1

[FIG. 2]: Fracture of the sugar-coated layer—Scanning Electron Microscopy x135. Sugar-coating liquid Man_pva_1

[FIG. 3]: Fracture of the sugar-coated layer—Scanning Electron Microscopy x135. Sugar-coating liquid Man_st.ac

FIG. 2

[FIG. 4]: Fracture of the sugar-coated layer—Scanning Electron Microscopy x1080. Sugar-coating liquid Man_1

[FIG. 5]: Fracture of the sugar-coated layer—Scanning Electron Microscopy x1080. Sugar-coating liquid Man_pva_1

[FIG. 6]: Fracture of the sugar-coated layer—Scanning Electron Microscopy x1080. Sugar-coating liquid Man_st.ac_1

FIG. 7

[FIG. 7]: Water uptake over time of coated tablets with different sugar-coating liquids, in an atmosphere at 30° C. and 75° C., at 5% sugar-coating.

FIG. 8

[FIG. 8]: Water uptake over time of tablets coated with different sugar-coating liquids, in an atmosphere at 30° C. and 75° C., at 10% sugar-coating.

FIG. 9

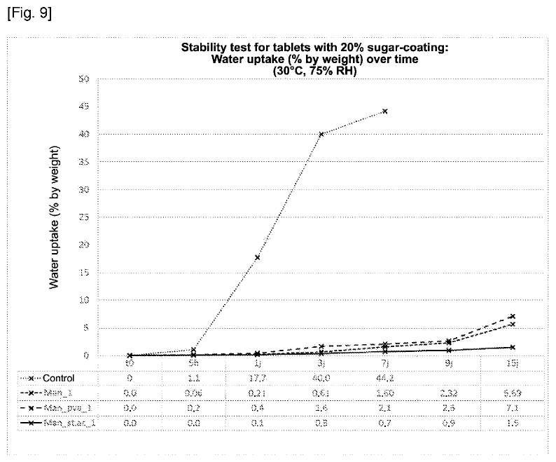

[FIG. 9]: Water uptake over time of tablets coated with different sugar-coating liquids, in an atmosphere at 30° C. and 75° C., at 20% sugar-coating.

### DETAILED DESCRIPTION OF THE INVENTION

Therefore, the object of the present invention is sugar-coated solid forms comprising at least one sugar-coating layer comprising mannitol and fatty acids.

In sugar-coating, the term “solid form” is conventionally understood to mean any solid presentation of sugar-coated substances (“sugar-coated solid form”) or capable of undergoing a sugar-coating operation (“core”). Typical examples are tablets, hard capsules, soft capsules, pellets, microspheres, granules, seeds, cookies, breakfast cereals, confectionery such as chewing gums, boiled candies, chewing candies, gummies, chocolates, fruits and vegetables, or products in the form of powders and/or crystals. These solid forms may, for example, be intended for food, pharmaceutical, veterinary or cosmetic use. They can be intended for humans, adults or children, or for animals. They may also be products intended for chemical or agrochemical use, although solid forms intended to be ingested in the context of the present invention are preferred. Preferably, these solid forms are selected from tablets.

Preferably, the sugar-coating layer in accordance with the invention comprises at least 40.0% mannitol, preferably at least 50.0%, preferably at least 60.0%, preferably at least 65.0%, preferably at least 70.0%, preferably at least 75.0%, preferably at least 80.0%, preferably at least 81.0%, preferably at least 82.0%; these percentages being expressed as dry weight of mannitol with respect to the total dry weight of said sugar-coating layer.

The fatty acids of the invention may or may not be in the form of salts without distinction. Preferably, the fatty acids of the sugar-coating layer in accordance with the invention are selected from saturated fatty acids. Preferably, the fatty acids of the invention are selected from fatty acids having 5 to 25 carbon atoms, preferably 10 to 20 carbon atoms, preferably 12 to 24 carbon atoms, preferably 14 to 22 carbon atoms, preferably 16 to 20 carbon atoms, for example, 16 to 18 carbon atoms. Preferably, the fatty acids of the invention comprise palmitic acid and/or stearic acid. Still preferably, the fatty acids useful in the invention comprise stearic acid, preferably at a content of at least 50% by dry weight with respect to the total dry weight of fatty acids. Preferably, this content is at least 70%, preferably at least 90%. Still preferably, stearic acid is the only fatty acid in the sugar-coating liquid.

Preferably, the sugar-coating layer in accordance with the invention comprises from 1.0 to 60.0% fatty acids, preferably from 1.0 to 40.0%, preferably from 2.0 to 30.0%, preferably from 5.0 to 30.0%, preferably from 10.0 to 20.0%, preferably from 12.0 to 18.0%, preferably from 14.0 to 16.0%, for example 15%; these percentages being expressed as dry weight of fatty acids with respect to the total dry weight of said sugar-coating layer.

Preferably, the sugar-coating layer in accordance with the invention comprises from 1.0 to 60.0% stearic acid, preferably from 1.0 to 40.0%, preferably from 2.0 to 30.0%, preferably from 5.0 to 30.0%, preferably from 10.0 to 20.0%, preferably from 12.0 to 18.0%, preferably from 14.0 to 16.0%, for example 15%; these percentages being expressed as dry weight of stearic acid with respect to the total dry weight of said sugar-coating layer.

Preferably, the object of the present invention is a sugar-coated solid form comprising at least one sugar-coating layer comprising:

40.0 to 99.0% by dry weight of mannitol;
1.0 to 60.0% by dry weight of fatty acids, these percentages being expressed as dry weight with respect to the total dry weight of said sugar-coating layer.

Preferably, the mannitol/fatty acid dry-weight ratio of the sugar-coating layer in accordance with the invention is less than or equal to 99, preferably less than or equal to 80, preferably less than or equal to 50, preferably less than or equal to 40, preferably less than or equal to 30, preferably less than or equal to 20, preferably less than or equal to 15, preferably less than 15, preferably less than or equal to 10. This ratio is preferably at least equal to 1, preferably at least equal to 2, preferably at least equal to 3, preferably at least equal to 4, for example selected from a range from 4 to 7, or from 5 to 7.

Preferably, when stearic acid is used, the mannitol/stearic acid dry-weight ratio of the sugar-coating layer in accordance with the invention is less than or equal to 99, preferably less than or equal to 80, preferably less than or equal to 50, preferably less than or equal to 40, preferably less than or equal to 30, preferably less than or equal to 20, preferably less than or equal to 15, preferably less than 15, preferably less than or equal to 10. This ratio is preferably at least equal to 1, preferably at least equal to 2, preferably at least equal to 3, preferably at least equal to 4, for example selected from a range from 4 to 7, or from 5 to 7.

Preferably, the sugar-coated solid forms of the invention have a percentage of sugar coating greater than 1%. This percentage of sugar-coating (% sugar-coating), also known as “mass gain”, is conventionally determined as follows:

$$\% sugarcoating = \frac{Sugar - coated solid form weight - Core weight}{Core weight} \times 100$$

Preferably, this percentage of sugar coating is greater than 3%, preferably greater than or equal to 4%, preferably greater than or equal to 5%. Preferably, the percentage of sugar coating is less than 60%, preferably less than 55%, preferably less than 50%, preferably less than 45%, preferably less than 40%, preferably less than 35%, preferably less than 30%, more preferably less than 25%. This mass gain is, for example, selected in a range from 3 to 30%, or in a range from 3 to 25%, or in a range from 5 to 20%.

The sugar-coating layer in accordance with the invention may comprise substances other than mannitol and fatty acids, as long as this does not contravene the properties sought in the present invention, in particular concerning the stability of the sugar-coated solid forms obtained. Such other compounds are, for example:

binders, such as gum arabic, polyvinyl alcohol (PVA), starch derivatives such as hydrolyzed hydroxypropylated starches;
crystallizable materials other than mannitol;
coloring substances such as pigments, opacifiers such as, for example, titanium dioxide or calcium carbonate;
flavorings, sweeteners;
active agents of interest, for example, pharmaceutical, nutritional, nutraceutical or phytosanitary agents.

Therefore, preferably, the object of the present invention is a sugar-coated solid form comprising at least one sugar-coating layer consisting of:

40.0 to 99.0% by dry weight of mannitol;
1.0 to 60.0% by dry weight of fatty acids;
0.0 to 49.0% by dry weight of other ingredients, in particular as defined hereinbefore; the sum of these percentages being equal to 100.0%.

However, preferably in the present invention, when binders are used, the mannitol/binders dry-weight ratio is preferably greater than 1, preferably greater than 2, preferably greater than 3, preferably greater than 5, preferably greater than 10, preferably greater than 15, preferably greater than 20, preferably greater than 30, preferably greater than 40, preferably greater than 50, preferably greater than 80, preferably greater than 99. Still preferably, the sugar-coating liquid useful in the invention is free of binders, at least free of binders of synthetic origin, and in particular of PVA.

The crystallizable materials other than mannitol are conventionally selected from sugars and polyols, conventionally selected from monomers and dimers, preferably from xylitol, sucrose, erythritol, dextrose, isomalt, maltitol or optionally a combination thereof. Preferably, however, the sugar-coating layer in accordance with the invention comprises less than 20.0%, preferably less than 10.0%, more preferably less than 5.0%, more preferably less than 1.0%, of xylitol, and/or sucrose, and/or erythritol, and/or isomalt, and/or maltitol, and/or sugars, and/or polyols other than mannitol; these percentages being expressed as dry weight of said substances with respect to the total dry weight of said sugar-coating layer.

Conventionally, the term “crystallizable materials” in sugar coating is understood to mean substances capable of crystallizing by evaporation of the solvent in which they are dissolved. It is these crystallizable materials that form the crystalline coating targeted by the sugar coating.

Generally, the sugar-coating layer in accordance with the invention comprises an amount of pigments and/or colorants less than 5.0%, preferably less than 4.0%, preferably less than 3.0%, preferably less than 2.0%, preferably less than 1.0%; these percentages being expressed as dry weight of pigments and/or colorants with respect to the total dry weight of said sugar-coating layer.

Generally, the sugar-coating layer in accordance with the invention comprises an amount of opacifiers, particularly titanium dioxide, less than 20.0%, preferably less than 18.0%, preferably less than 15.0%, preferably less than 10.0%, preferably less than 5.0%, preferably less than 4.0%, preferably less than 3.0%, preferably less than 2.0%, preferably less than 1.0%; these percentages being expressed as dry weight of opacifiers with respect to the total dry weight of said sugar-coating layer. In an advantageous embodiment, the sugar-coating layer of the sugar-coated solid forms of the invention is free of titanium dioxide, or more generally of opacifiers. In the case where the sugar-coating layer is free of opacifier or titanium dioxide, the sugar-coated solid form of the invention is preferably white.

Another object of the invention is a sugar-coating method, particularly useful for the preparation of sugar-coated solid forms in accordance with the invention, comprising

a step (a) of spraying a sugar-coating liquid onto a bed of moving cores, said cores being placed in a chamber equipped with a perforated rotary drum, and said spraying being ensured by means of at least one compressed air nozzle;
at least one step (b) concomitant with step (a) of drying said sprayed sugar coating liquid;
a step (c) of collecting the resulting sugar coated solid forms; said sugar-coating liquid comprising mannitol and fatty acids.

The equipment used to implement the method of the invention typically comprises a unit for storing the sugar-coating liquid, comprising at least one outlet for transporting the sugar-coating liquid to a device for spraying the sugar-coating liquid. The sugar-coating liquid is applied, via the spraying device, to a bed of cores contained in the chamber, said chamber being provided with a rotary drum for moving said bed of cores. More precisely, the drum is a perforated rotary drum, and the selected spraying device comprises at least one compressed air nozzle. The equipment further comprises an air inlet at the drum chamber for drying the sugar-coating liquid. The drying air is discharged in particular through the bed of cores via the perforations of the rotary drum, in particular by suction of the air from the chamber.

The elements of the equipment useful for the invention are commercially available, and their arrangement does not represent particular difficulties for the person skilled in the art.

The sugar-coating liquid useful in the invention comprises mannitol and a fatty acid. Preferably, the sugar-coating liquid of the invention has a dry material whose composition is as disclosed above, for the sugar-coated solid form of the invention.

Typically, in particular when the sugar-coating liquid is an aqueous liquid, the sugar-coating liquid of the invention has a concentration of crystallizable materials, in particular mannitol, of less than 85%, this percentage corresponding to the dry weight of crystallizable materials of the sugar-coating liquid with respect to the total weight of said sugar-coating liquid. Preferably, this concentration is less than or equal to 70%, preferably less than or equal to 60%, preferably less than 60%, even less than or equal to 55%, even less than or equal to 50%, even less than or equal to 45%, even less than or equal to 40%, even less than or equal to 35%, even less than or equal to 30%, even less than or equal to 25%, even equal to 20%. This concentration is generally greater than 10%, or even at least equal to 15%. This concentration is selected, for example, from a range from 16 to 18%.

Preferably, the sugar-coating liquid of the invention has a dry material content by weight of less than 85%, preferably less than 80%, preferably less than 75%, preferably less than or equal to 70%, preferably less than or equal to 60%, preferably less than 60%, even less than or equal to 55%, even less than or equal to 50%. This dry material is generally greater than 10%, preferably at least equal to 15%, more preferably at least equal to 18%. This dry material is, for example, selected from a range from 15 to 50%, preferably from 18 to 40%, preferably from 18 to 35%. As can be seen from the examples below, this dry material may well be less than 35%, even less than or equal to 30%, even less than or equal to 25%, for example, in a range of 18 to 22%.

The sugar-coating liquid used in accordance with the invention is typically polar, and preferably comprises water as the majority solvent, most preferentially as the sole solvent.

In an advantageous embodiment, for reasons of simplicity and since this is possible, the sugar-coating method according to the invention implements a single sugar-coating liquid, that is to say the sugar-coating liquid has a constant formulation throughout the duration of the sugar-coating.

The temperature of the sugar-coating liquid is typically selected so that the crystallizable materials, in particular mannitol, are well solubilized in said sugar-coating liquid to be sprayed. This temperature is thus also a function of the amount of crystallizable materials present in the liquid. In the method of the invention, this temperature is typically selected in a range from 20 to 90° C. Preferably, this temperature is less than 85° C., preferably less than 80° C., preferably less than 75° C., preferably less than 70° C., even less than 65° C., even less than 60° C., even less than 55° C., even less than 50° C., even less than 45° C., even less than 40° C., even less than 35° C., even less than 30° C. This temperature is generally at least 15° C., even at least 20° C. It corresponds, for example, to room temperature, which typically ranges from 20 to 25° C.

In an advantageous embodiment, the sugar-coating liquid is stored in a single jacket storage unit, and/or the equipment useful in the method of the invention does not have a device for heating the sugar-coating liquid. Indeed, since the method of the invention does not necessarily require the use of high temperatures of sugar-coating liquids, these devices are not obligatory in the method of the invention.

For spraying the sugar-coating liquid, the number of compressed air nozzles used is conventionally selected based on the dimensions of the sugar-coating chamber, according to the manufacturer's recommendations. This number of nozzles is typically from 1 to 2 nozzle(s) per 40 cm diameter section of the sugar-coating chamber. This number of nozzles ranges, for example, from 1 to 10, for example from 1 to 6.

Preferably, for spraying, the method of the invention uses only compressed air nozzles.

Preferably, the nozzles used in accordance with the invention have a hole having a diameter selected from a range from 0.1 to 2.8 mm, preferably from 0.1 to 2.5 mm, preferably from 0.1 to 2.2 mm, for example from 0.3 to 2.0 mm, or from 0.5 to 1.8 mm or from 0.5 to 1.5 mm, or from 0.5 to 1.2 mm, or from 0.5 to 1.0 mm.

Preferably, the spray flow rates are selected from a range from 0.5 to 20.0 g/min/kg of cores, preferably from 1.0 to 20.0 g/min/kg of cores, preferably from 2.0 to 20.0 g/min/kg of cores, for example, from 2.0 to 15.0 g/min/kg of cores, or even from 2.0 to 10.0 g/min/kg of cores.

In an advantageous embodiment, the flow rate selected for the spraying is increased during the sugar-coating method. In particular, the inventors have found that, for certain crystallizable materials, the use of low flow rates at the beginning of sugar coating makes it possible to promote the first crystallization phase which takes place on the surface of the cores. The flow rate can then be increased so as to accelerate the sugar coating.

For spraying, the atomization pressure and collapse pressure are adjusted based on the flow rate and the hole of the nozzle, and in accordance with the manufacturer's recommendations. This flow rate and this nozzle hole typically depend on the size of the equipment used. These atomization and collapse pressures are typically selected in a range from 0.5 to 4.0 bar, preferably from 0.5 to 3.5 bar, for example from 0.7 to 2.5 bar for the atomization pressure, and/or from 0.7 to 3.5 bar for the collapse pressure.

In the present invention, the sugar-coating liquid is sprayed onto a bed of cores moved by means of a rotary drum.

The nature of these cores is preferably as defined above for solid forms;

they are, for example, tablets or chewing gums. These cores may be completely bare, or may be coated with one or more layers, for example gumming, film-coating or even sugar-coating layers, said layers preferably being obtained in the same equipment as that used for the sugar-coating method of the invention.

To set the bed of cores in motion, the speed of rotation of the drum is selected based on the dimensions of the chamber and of the size of the cores to be sugar coated. It is generally selected in a range from 3 to 30 rpm, for example, in a range from 10 to 20 rpm.

In an advantageous embodiment, in particular for reasons of simplicity and/or space, and because the method of the invention allows it, the moving of the bed of cores excludes the transport of said cores along a longitudinal axis. This means in particular that the cores being sugar-coated are not transported from one chamber to another, that is to say that they are sugar-coated in a single chamber, and/or that the bed of cores is not sugar-coated in a longitudinal chamber along which they are transported.

For drying, the temperature selected for the drying air is preferably less than 100° C., preferably less than or equal to 80° C., preferably less than or equal to 75° C., preferably less than or equal to 70° C., even less than or equal to 65° C., even less than or equal to 60° C., even less than or equal to 55° C., even less than or equal to 50° C., even less than or equal to 45° C., even less than or equal to 40° C., even less than or equal to 35° C., even less than or equal to 30° C., even less than or equal to 25° C. This drying temperature is generally at least 15° C., preferably at least 20° C.

For drying, the flow rate may be selected in a range from 50 to 8,000 m³/h, for example, in a range from 100 to 7,000 m³/h, for example, from 100 to 1,000 m³/h.

The outlet of the drying air is advantageously carried out by suction of air by means of the perforations of the perforated rotary drum.

Preferably, the perforated wall area of the rotary drum useful in the invention preferably represents at least 50% of the surface of said wall of the drum, preferably at least 60%, preferably at least 70%, preferably at least 80%, preferably at least 90%. Most preferentially, the wall of the drum is perforated over its entire surface.

Preferably, the temperature of the bed of cores being sugar-coated is at most 70° C., preferably at most 60° C., preferably at most 55° C., preferably at most 50° C., preferably at most 45° C., preferably at most 40° C. This temperature of the bed of cores being sugar-coated is generally at least 10° C., even at least 15° C., even at least 20° C., even at least 25° C. This temperature of the bed of cores being sugar-coated is preferably in a range from 30 to 40° C., preferably in a range from 32 to 38° C., for example, about 35° C.

Preferably, before the beginning of the sugar-coating, that is to say before the spraying begins, the method of the invention comprises a step of heating the core bed to be sugar-coated. This step aims in particular at bringing the core bed to a target temperature which corresponds to that of the cores being sugar-coated.

The sugar-coating method of the invention concomitantly sprays and dries the spraying liquid.

However, it is conceivable to introduce distribution (“pause time”) and spraying steps in the absence of drying, as long as this does not contravene the properties sought in the present invention, in particular relating to the quality of the sugar-coated solid forms obtained and/or the workability of the method.

Preferably, the phases of the sugar-coating method, during which spraying is optionally not carried out concomitantly with drying, represent less than 50% of the sugar-coating method time, preferably less than 40%, preferably less than 30%, preferably less than 20%, preferably less than 10%, preferably less than 5%. Most preferentially, the sugar coating does not comprise a spraying phase in the absence of drying.

Preferably, the possible pause times (time between two spraying phases during which there is neither spraying nor drying) represent less than 50% of the time of the sugar-coating method, preferably less than 40%, preferably less than 30%, preferably less than 20%, preferably less than 10%, preferably less than 5%. Most preferentially, the sugar coating does not comprise a pause time.

Preferentially, the percentage of sugar-coating (or “mass gain”) is, per unit of time, at least 0.05% per minute, preferably at least 0.10% per minute, preferably at least 0.13% per minute, or even at least 0.15% per minute.

The method of the invention may further comprise other usual steps than those aimed at sugar-coating the solid forms, as long as this does not contravene the properties sought in the present invention, in particular related to the quality of the sugar-coated solid forms obtained and/or the workability of the method. Examples of other steps which may be mentioned include gumming, smoothing, polishing and coloring. Advantageously, if such steps are carried out, they are carried out in the same equipment as that used in the sugar-coating method of the invention. It is understood that some of these steps, such as, for example, gumming or coloring, may sometimes be assimilated to sugar-coating steps, if the composition used comprises such an amount of crystallizable materials that a layer of crystallized substances, in particular sugars and/or polyols, is effectively formed.

With regard to these other usual steps, it is interesting to note that the sugar coating carried out according to the method of the invention already makes it possible to obtain a good level of smoothing of the solid forms. The coloring may also be carried out directly via adding coloring agents in the sugar-coating liquid.

The method of the invention may also comprise a priming step. This priming step consists of applying, once or several times, a powdery composition on the bed of cores to be sugar-coated, or during sugar-coating, preferentially during sugar-coating. This powdery composition typically comprises crystalline materials. These crystalline materials are typically of the same nature as the crystallizable materials of the sugar-coating liquid, but may be different. The amount of powdery composition applied is typically less than 10% by weight with respect to the weight of the tablets, preferably less than 5%. Preferably, however, and since this is not necessary in the method of the invention, the latter does not implement a priming step.

The equipment useful for the invention may comprise other elements conventionally used in sugar-coating method, as long as this does not contravene the properties sought in the present invention, in particular related to the quality of the sugar-coated solid forms obtained and/or the workability of the method.

In this respect, it is interesting to note that the background art sometimes mentions the use of lump breakers, the purpose of which is to avoid the appearance of sticking during the sugar coating. Such lump breakers are not necessary in the method of the invention. Thus, preferably, and because it is advantageously possible in the context of the method of the invention, the chamber of the drum used in accordance with the invention is free of lump breakers.

An object of the present invention is also the use of a combination of mannitol and fatty acids for coating solid forms in order to improve the stability of said solid forms.

Preferably, the fatty acids are selected from those as defined above for the sugar-coating layer comprising mannitol and fatty acids of the sugar-coated solid forms of the invention.

Preferably, the mannitol/fatty acid dry-weight ratio of said combination used is as disclosed above for the sugar-coating layer comprising mannitol and fatty acids of the sugar-coated solid forms of the invention.

The use to improve the stability of the solid forms may also comprise the use of substances other than the mannitol/fatty acid combination useful for the invention, as long as this does not contravene the properties sought in the present invention, in particular related to the stability of the said solid forms; these other substances being preferably as disclosed above for the sugar-coating layer comprising mannitol and fatty acids of the sugar-coated solid forms of the invention, in relation to their nature and the amounts employed.

Preferably, the coating in which the mannitol/fatty acid combination is used is a sugar-coating layer, preferably as disclosed above for the sugar-coating layer comprising mannitol and fatty acids of the sugar-coated solid forms of the invention.

Preferably, the mannitol/fatty acid combination is used in a sugar-coating method, preferably in the sugar-coating liquid, preferably in accordance with the sugar-coating method of the invention as disclosed above.

Preferably, the mannitol/fatty acid combination useful in the invention is used to improve the stability of the solid forms with respect to humidity.

This ability to improve the stability of the solid forms with respect to humidity can be easily determined by the skilled person by comparing the water uptake by weight of solid forms coated by means of the mannitol/fatty acid combination of the invention with these same uncoated solid forms, after a determined storage time. For the stability test, it is possible, for example, to place the solid forms in an environment at 75% relative humidity, at a temperature of 30° C. (t0). The water uptake by weight is then determined over time at t0+5 h, and/or at t0+1 day, and/or at t0+3 days, and/or at t0+7 days, and/or at t0+9 days, and/or at t0+15 days. It is possible, for example, to proceed according to the Examples below.

Preferably, the mannitol/fatty acid combination useful in the invention is used to improve the stability of the solid forms in an atmosphere comprising at least 60% relative humidity, preferably at least 65%, preferably at least 70%, preferably at least 75%.

Preferably, the mannitol/fatty acid combination useful in the invention is used to improve the stability of the solid forms in an atmosphere of at least 15° C. temperature, preferably at least 20° C., preferably at least 25° C., preferably at least 30° C.

Preferably, the mannitol/fatty acid combination useful in the invention is used to decrease the water uptake of the solid forms as compared with these same uncoated solid forms (control). Preferably, this decrease in water uptake is determined under the relative humidity and/or temperature conditions as given above. Preferably, this decrease in water uptake is determined at the times as listed above. Preferably, this decrease in water uptake is at least 30%, preferably at least 40%, preferably at least 50%, preferably at least 60%, preferably at least 65%, preferably at least 70%, preferably at least 75%, preferably at least 80%, preferably at least 85%, preferably at least 90%, preferably at least 95%, even at least 96%, even at least 97%, even at least 98%, even at least 99%.

In particular, this decrease in water uptake is calculated as follows:

$$\% decrease in water uptake = \frac{{WU}_{c} - {WU}_{T}}{{WU}_{c}} \times 100$$

where:
WU_{C} “water uptake, control,” means the mass gain obtained with the control solid forms;
WU_{T} “water uptake, test” means the mass gain obtained with the coated solid forms to be tested.

Preferably, the mannitol/fatty acid combination useful in the invention is used to improve the stability of the solid forms comprising humidity-sensitive substances. The term humidity-sensitive substances is conventionally understood to mean so-called hygroscopic substances, that is to say substances having a tendency to absorb humidity, and/or substances whose activity is impaired by humidity (for example pharmaceutical active ingredients whose pharmacological activity is changed by the presence of water).

In the present invention, it is understood that when reference is made to a dry mass of compounds, this mass includes possible impurities, and that reference is indeed made to a weight of anhydrous substance.

The invention will be better understood with the aid of the following examples, which are intended to be illustrative and non-limiting.

### EXAMPLES

1. Material and Method

1.1 Equipment Used

Equipment with the following characteristics was used:

Perforated rotating drum: 30.48 cm diameter, 2 L volume;

Anti-slip bars x6;

1 compressed air atomization nozzle (Schlick 970/7-1 S75); 0.8 mm hole diameter;

Peristaltic pump (Watson Marlow model 323); 3 roller head (313 DW);

Tube: 2 mm internal diameter: 6 mm external diameter.

1.2 Sugar-Coating Liquids

The sugar-coating liquids tested had the following formulations (% dry):

TABLE 1

| Ref. | Man_1 | Man_pva_1 | Man_st.ac_1 | Man_st.ac_2 | Man_st.ac_3 |
|---|---|---|---|---|---|
| Mannitol | 98.5% | 83.5% | 83.5% | 54.7% | 54.7% |
| PVA (polyvinyl | — | 15.0% | — | — | — |
| alcohol) | | | | | |
| LYCOAT ® RS720* | — | — | — | 16.4% | 16.4% |
| Stearic acid | — | — | 15.0% | 10.9% | 10.9% |
| Titanium dioxide | 1.0% | 1.0% | 1.0% | 17.5% | — |
| Calcium carbonate | — | — | — | — | 17.5% |
| Polysorbate 80 | — | — | — | 0.2% | 0.2% |
| (TWEEN 80) | | | | | |
| Blue dye (lake) | 0.5% | 0.5% | 0.5% | 0.3% | 0.3% |
| Mannitol/PVA or | — | 6 | 6 | 5 | 5 |
| mannitol/stearic | | | | | |
| acid dry-weight ratio | | | | | |
| Dry material | 20% | 20% | 20% | 32% | 32% |

*Hydrolyzed (or “fluidized”) hydroxypropylated pea starch.

All these sugar-coating liquids were contained in a single-jacket storage tank, and at room temperature (20-25° C.).

It should be noted that sugar-coating liquids in accordance with the invention and free of opacifying agents have also been tested, although the results are not presented herein. Interestingly, the solid forms thus sugar-coated, in particular those of white color, do not yellow over time. It is therefore possible to dispense with these opacifying agents.

1.3 Cores

The following batches of cores were used as the sugar-coating substrate: 900 g sorbitol tablets, 380 mg (hygroscopic).

1.4 Sugar-Coating

Several tests have been carried out so as to vary the mass gain of the sugar-coated solid forms.

Before starting spraying, the tablet bed was heated in order to reach the target temperature (in this case 35° C.) for the bed of cores (approximately 2 minutes in the present case). Spraying and drying were then carried out concomitantly throughout the method (no pause time). The sugar-coating liquid was at room temperature (20-25° C.). At the end of the sugar-coating, the spraying was stopped, the turbine slowed down, and the solid forms were dried until cooling to a target temperature (about 2 minutes).

The conditions for implementing the method were as follows: Operating pressure: 4 bar; Atomization pressure: 1.3 bar; Jet collapse pressure: 1.3 bar; Differential pressure: −0.25 mbar; Drum spinning speed: 18 rpm; Drying air flow rate: 100 m³/h; Inlet air temperature (drying): 55° C.; Measured core bed temperature: 35° C. (this temperature has moreover been controlled so as to adjust, if necessary, the drying temperature and/or of the spray flow rate. In this case, no such adjustment was required); Spray Flow Rate and Method Time:

TABLE 2

| | For 5% | | For 10% | | For 20% | |
|---|---|---|---|---|---|---|
| | sugar- | | sugar- | | sugar- | |
| | coating | | coating | | coating | |
| Total volume “X” | 250 | g | 500 | g | 1,000 | g |
| of solution sprayed | | | | | | |
| Spray flow rate at the | 3.5 | g/min | 3.5 | g/min | 3.5 | g/min |
| beginning, 0 to 50 g of | | | | | | |
| sprayed solution | | | | | | |
| Spray flow rate from | 7.5 | g/min | 7.5 | g/min | 7.5 | g/min |
| 50 g to X g | | | | | | |
| Sugar-coating time | 50 | min | 85 | min | 150 | min |

2. Results

2.1 Appearance of the Coated Solid Forms

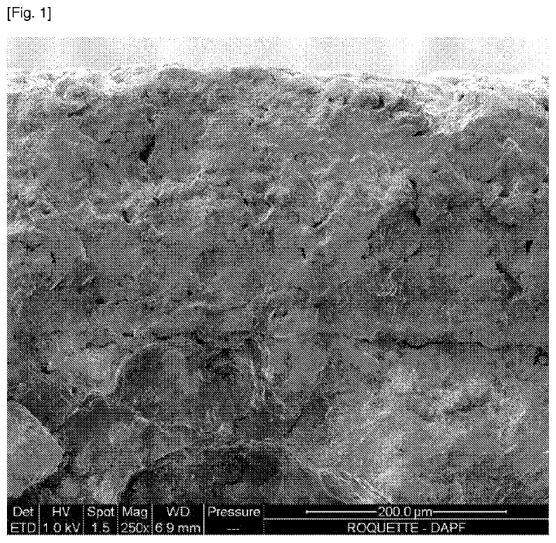

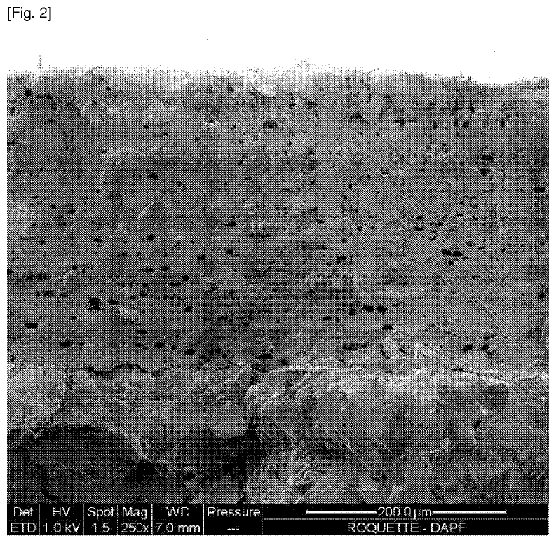

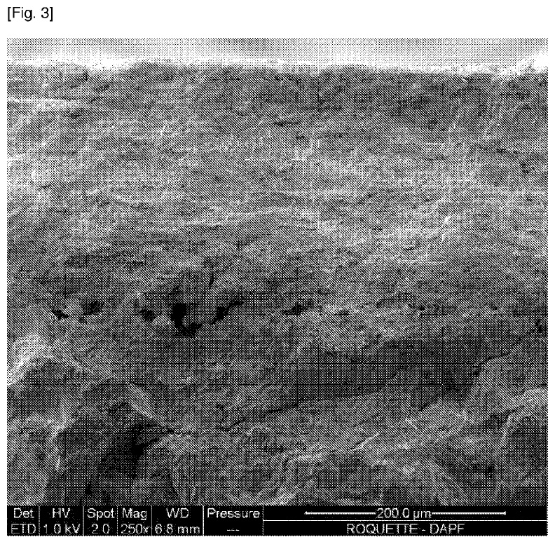

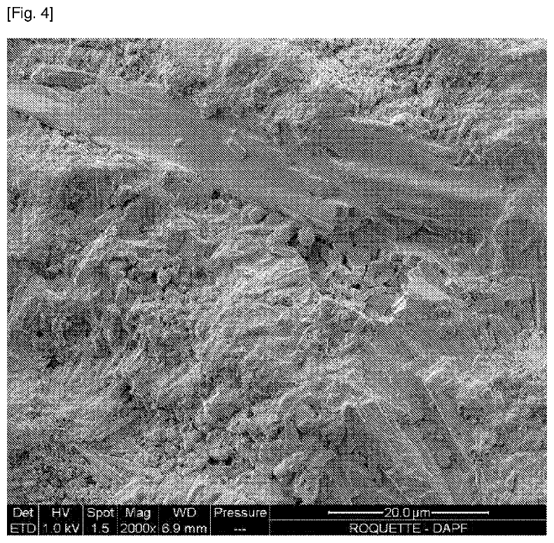

The solid forms obtained with the control sugar-coating liquids Man_1, Man_pva_1 and Man_st.ac_1, having a sugar-coating percentage of 20%, were observed under electron microscopy. The photographs are shown in FIGS. 1 to 6.

2.2. Stability Tests

Some of the tablets obtained in item 1 were placed in an atmosphere at 30° C., at 75% relative humidity (t0). As a control of instability, bare (uncoated) sorbitol tablets were also tested.

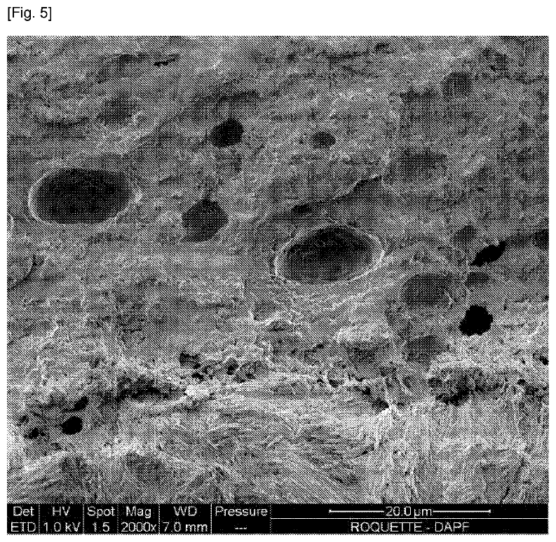

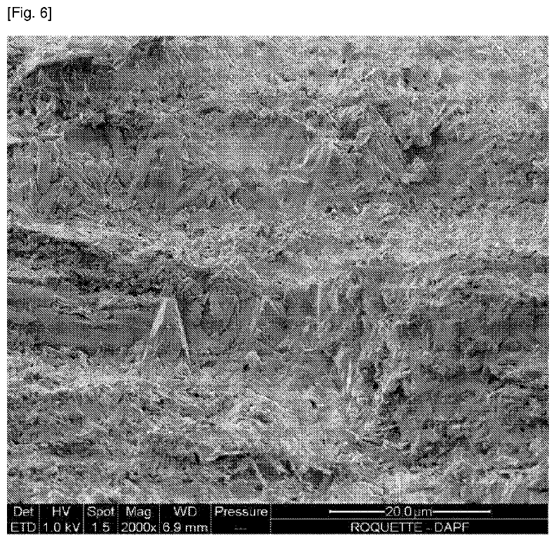

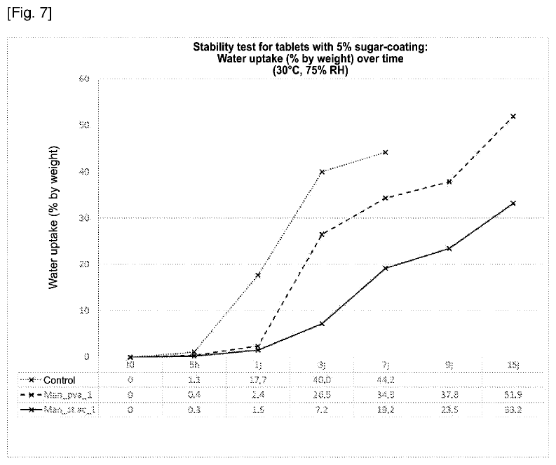

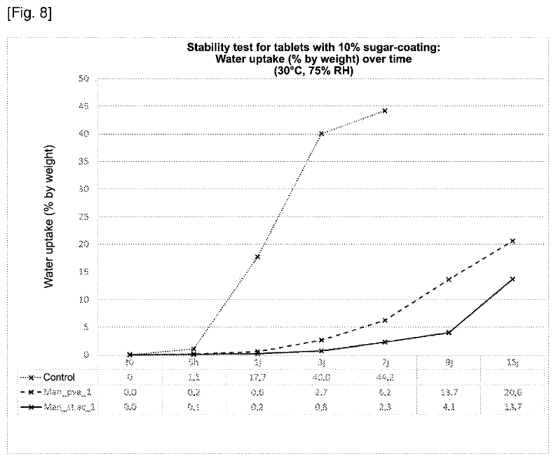

The water uptake of the tablets was evaluated at t0+5 h, t0+1 day, t0+3 days, t0+7 days, t0+9 days, t0+15 days. The results are presented in FIGS. 7, 8 and 9, for a sugar-coating percentage of 5%, 10% and 20% respectively. In accordance with the teaching of U.S. Pat. No. 8,846,089, it is observed that the solid forms coated with mannitol and PVA have improved stability: compared with the control, the uptake of water over time of the coated solid forms is markedly reduced.

Furthermore, whatever the composition used for the coating, the comparison of FIGS. 1 to 7 shows that the higher the percentage of sugar coating, the greater the stability.

Whatever the mass gain (from 5% to 20%), the stability of the sugar-coated solid forms of the invention comprising mannitol and fatty acids (stearic acid) is even greater. The water uptake over time of the sugar-coated solid forms of the invention is markedly less than that observed with the control solid forms or solid forms coated with mannitol and PVA, from t0+5 h to t0+15 days. For the highest percentage of sugar coating (20%, FIG. 7), the inventors have also tested a sugar-coating with mannitol alone (sugar coating liquid “Man_1”), according to a method which has been the subject of a French patent application No. 1755557 in the name of the applicant. This patent application, not yet published, discloses a method similar to that of the present invention, with the exception of the fact that no mention is made of the use of fatty acids.

It is found that the addition of PVA has only a minor impact on the improvement of stability, while the addition of stearic acid allows a clearly visible decrease in water uptake.

Thus, the sugar coating carried out in accordance with the present invention makes it possible to obtain an excellent protection of the solid forms, in particular with respect to humidity absorption.

1. A sugar-coated solid form, wherein said sugar-coated solid form comprises at least one sugar-coating layer comprising mannitol and fatty acids.

2. The sugar-coated solid form according to claim 1, wherein said sugar-coating layer comprises at least 40.0% of mannitol; this percentage being expressed as dry weight of mannitol with respect to the total dry weight of said sugar-coating layer.

3. The sugar-coated solid form according to claim 2, wherein said sugar-coating layer comprises at least 50.0% of mannitol; this percentage being expressed as dry weight of mannitol with respect to the total dry weight of said sugar-coating layer.

4. The sugar-coated solid form according to claim 1, wherein said sugar-coating layer comprises from 1.0 to 60.0% of fatty acids; this percentage being expressed as dry weight of fatty acids with respect to the total dry weight of said sugar-coating layer.

5. The sugar-coated solid form according to claim 4, wherein said sugar-coating layer comprises from 1.0 to 40.0% of fatty acids; this percentage being expressed as dry weight of fatty acids with respect to the total dry weight of said sugar-coating layer.

6. The sugar-coated solid form according to claim 1, wherein the mannitol/fatty acid dry-weight ratio of said sugar-coating layer is less than or equal to 99.

7. The sugar-coated solid form according to claim 6, wherein the mannitol/fatty acid dry-weight ratio of said sugar-coating layer is less than or equal to 80.

8. The sugar-coated solid form according to claim 7, wherein the dry-weight ratio of mannitol/fatty acids of said sugar-coating layer is less than or equal to 50.

9. The sugar-coated solid form according to claim 1, wherein said sugar-coating layer further comprises at least one binder.

10. The sugar-coated solid form according to claim 9, wherein the mannitol/binder dry-weight ratio of said sugar-coating layer is greater than 1.

11. A method of sugar-coating solid forms, wherein said method comprises:
a step (a) of spraying a sugar-coating liquid onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid;
a step (c) of collecting the resulting sugar-coated solid forms; said sugar-coating liquid comprising mannitol and fatty acids.

12. The method of sugar-coating solid forms according to claim 11, wherein said sugar-coating liquid has a concentration of crystallizable materials, in particular mannitol, of less than 85%, this percentage corresponding to the dry weight of crystallizable materials of the sugar-coating liquid with respect to the total weight of said sugar-coating liquid.

13. The method of sugar-coating according to claim 11, wherein said sugar-coating liquid has a dry material content by weight of less than 85%.

14. Use of a combination of mannitol and fatty acids for sugar-coating solid forms to improve the stability of said solid forms.

15. The use according to claim 14, for improving the stability of solid forms against humidity.
